(19) United States
(12) Patent Application Publication
(10) Pub. No.: US 2022/0185540 A1
(43) Pub. Date: Jun. 16, 2022

(54) INSTRUMENT TRAY INDICATOR SYSTEM

(71) Applicant: Joint Development, LLC, Salt Lake, UT (US)

(72) Inventors: Sylvia Sorensen, Salt Lake City, UT (US); Eric M. Dacus, Salt Lake City, UT (US); Zachary Kay, Park City, UT (US); Nick Taylor, Salt Lake City, UT (US)

(21) Appl. No.: 17/553,616

(22) Filed: Dec. 16, 2021

Related U.S. Application Data
(60) Provisional application No. 63/126,391, filed on Dec. 16, 2020.

(51) Int. Cl. B65D 25/20; A61B 50/33

(52) U.S. Cl. CPC B65D 25/205; A61B 2050/3008; A61B 50/33

(57) ABSTRACT

The instrument tray indicator system includes a three-dimensional indicia associated with a least a portion of an instrument tray carrying at least one medical component. The three-dimensional indicia has a structure symbolic of the at least one medical component within the instrument tray, and the three-dimensional indicia is of a size and shape to provide external tactile feedback of the structure of the three-dimensional indicia through a sterile wrapping sealing an interior of the instrument tray with the at least one medical component therein. This allows one to identify the at least one medical component within the instrument tray without removing the sterile wrapping.

### BACKGROUND OF THE INVENTION

The present invention generally relates to an instrument tray indicator system. More specifically, the instrument tray indicator system includes indicia associated with an exterior of an instrument tray that includes a structure symbolic of a medical component therein for providing external tactile feedback regarding the identification of said medical component through sterile wrap enclosing the instrument tray prior to surgery.

Surgical instruments are tools or devices frequently used in, e.g., hospitals, physician offices, clinics, outpatient centers, urgent care or other emergency facilities, and may be used in a relatively wide variety of surgical fields such as general surgery, orthopedics, dentistry, veterinary clinics, etc. Surgical instruments are typically stored and transported in instrument trays and are delivered to the operating room for use by surgeons during an operation. For example, some surgical instruments may help or aid surgeons perform certain surgical operations, while other surgical instruments stored and transported within these trays may be used to help or aid the patient (e.g., implants designed to replace or repair damaged or worn out joints).

Of course, a large variety of surgical instruments have been designed over the years for use in connection with various surgical procedures, which vary in scope and complexity. As such, surgical instruments known in the art can vary greatly in size, shape, and form, depending on the type and nature of the surgery in which the surgical instruments are to be used. For example, in orthopedics, surgical instruments may be designed specifically to aid in surgical procedures that help correct or repair deformities or injuries to bones, ligaments, tendons, muscles, joints, etc. Surgical instruments that may be used by an orthopedic surgeon during such procedures may include scalpels, dissecting scissors, thumb forceps, retractors, mallets, elevators, probes, clamps, etc. Additionally, orthopedic surgical procedures also often require the use of surgical instruments in the form of implants, such as femoral components, patellar components, tibial components, femoral stems, heads, inserts, acetabular cups, etc., which may be used to correct or repair injured or worn out joints.

Important in this respect is that these surgical instruments are commonly stored in trays, which generally provide protection, organization, and a method for transporting the surgical instruments stored therein in and among locations within a medical facility, such as the operating room, sterilization facilities, and/or storage during non-use. Holding or retaining surgical instruments within the tray not only helps prevent movement therein during transportation that may cause damage to the surgical instruments, but also helps with organization, surgical workflow, preventing loss, and shortening the time it takes medical personnel to count inventory.

Of course, as with the surgical instruments themselves, there are a wide variety of surgical instrument trays known in the art, each of which may vary in size, shape, and/or configuration depending on the nature of the surgical procedure and/or the surgical instruments needed during the procedure. For more general surgical procedures, the surgical instrument tray may be configured to store a general set of operating instruments that may be used in a wide variety of surgical procedures. Such surgical instrument trays may include only general specifications and/or layouts; and have a generic size and shape such as a rectangular standard stainless-steel pan. Such basic trays may include minor differences that vary by manufacturer, medical facility requirements, and/or surgeon personal preference.

More advanced and/or complex surgical instrument trays may include a more ornate arrangement of internal couplings designed to hold certain surgical instruments in place in a specific series, orientation, or sequence therein. Some surgical instrument trays may also have a more rigid or robust outer body frame designed to hold multiple sets of surgical instruments, some of which may require sterilization after a surgery. As such, these surgical trays may also be designed for repeat exposure to high temperature environments during post-surgery sterilization procedures.

For more specialized surgical procedures such as orthopedic surgery (e.g., knee or hip replacement), the exact size, shape, and/or configuration of the surgical instrument tray may be important and may be specific the manufacturer of the joint implants. For example, surgical instrument trays for use in orthopedic surgeries typically include some form of layout based on the procedural workflow of the operation, i.e., the surgical instruments are typically stored in multiple instrument trays organized in a manner where the instruments are removed and used according to the sequence of the surgical procedure. Brackets and/or other retaining assemblies may be configured to ensure that the surgical instruments can only couple to these trays in certain locations to prevent accidental loading, thereby mitigating situations where surgical instruments are accessed or used in the wrong order during surgery. As such, this may help organize and streamline the orthopedic surgery to increase operating room efficiency and accuracy.

But, since orthopedic surgical trays are oftentimes all the same size, it can be difficult, if not impossible, to identify which trays hold certain medical instruments to ensure that the contents of the instrument trays are accessed in the correct order during surgery. In this respect, operating room technicians tend to spend a considerable amount of time preparing for surgery, including ensuring that the instrument trays are organized correctly and in the manner desired by the surgeon. While some instrument tray designs known in the art may include some level of generic indicia (e.g., one or more circles on the side), such indicia are typically formed integral with the tray (i.e., not being removable, swappable, or replaceable) and also fail to convey any sort of information regarding the actual contents of the surgical tray. In other words, the operating room technician may need to memorize which dot corresponds to which tray in advance of surgery. Even then, such a system is especially prone to human error given that the surgical trays all tend to have similar, if not the same, size and/or shape. This may be especially so since it is difficult to readily or quickly differentiate the contents of one tray from another when the trays are wrapped in sterile wrapping, thereby concealing the one or more circles formed from the side of the instrument tray.

There exists, therefore, a need in the art for an instrument tray indicator system that includes indicia formed thereon or therein in the form of geometric shapes, alphanumeric characters, or other symbols that enable medical professionals to identify the contents of an instrument tray wrapped in sterile wrapping by way of tactile feedback perceivable when wearing latex gloves. Such instrument tray indicator systems may be removable, replaceable, and/or swappable depending on the contents of the instrument tray. The present invention fulfills these needs and provides further related advantages.

### SUMMARY OF THE INVENTION

In one embodiment, an instrument tray indicator system as disclosed herein may include a three-dimensional indicia (e.g., an embossed number, letter, geometric shape, character, etc.) associated with at least a portion of an instrument tray carrying at least one medical component. The three-dimensional indicia may have a structure symbolic of the at least one medical component within the instrument tray and its size and shape may provide external tactile feedback of the structure of the three-dimensional indicia through a sterile wrapping sealing an interior of the instrument tray with the at least one medical component therein, thereby allowing one to identify the at least one medical component within the instrument tray without removing the sterile wrapping.

In one embodiment, the instrument tray may be a multi-sided enclosure and the three-dimensional indicia may be associated with at least one side of the multi-sided enclosure. Here, the multi-sided enclosure may include at least one recessed panel with the three-dimensional indicia attached thereto. The recessed panel may have a depth relatively greater than a thickness of the three-dimensional indicia so the three-dimensional indicia remains in non-contact relation with the sterile wrapping when tented over the recessed panel.

In another embodiment, the three-dimensional indicia may couple to at least one perforation formed in the multi-sided enclosure. Here, the three-dimensional indicia may selectively removably attach to the instrument tray, or may more permanently attach to the at least one perforation formed in the multi-sided enclosure (e.g., by way of a rivet or the like). Moreover, multiple of the three-dimensional indicia may be carried by a baseplate that selectively attaches to the instrument tray (e.g., such as by way of one or more of the perforations). In one embodiment, each of the multiple three-dimensional indicia on the baseplate may be arranged to generally align with a different row of medical components within the interior of the instrument tray. Here, each of the multiple three-dimensional indicia may be of a different structure symbolic of a different medical component within each different row. Alternatively, each of the multiple three-dimensional indicia may be arranged in a grid matrix representative of a layout of the different medical components within the instrument tray, wherein each of the multiple three-dimensional indicia may be of a different structure symbolic of a different medical component in the grid matrix.

In another embodiment, the three-dimensional indicia may be a circle symbolic of a femoral component, and may have a thickness between 0.05 and 1.0 inches. In other embodiments, the three-dimensional indicia may be a cutout formed within a sidewall of the instrument tray. In this latter embodiment, the thickness of the three-dimensional indicia may be that of the thickness of the instrument tray sidewall. Furthermore, the three-dimensional indicia may further include an edge break that includes a chamfer, a fillet, a radius, or a bevel to reduce potential abrasion in the event the three-dimensional indicia comes into contact with the sterile wrapping. Furthermore, the three-dimensional indicia may further be associated with at least one of a perforated sidewall, a front panel, a rear panel, a lid, or a bottom surface of the instrument tray.

In another embodiment, an instrument tray indicator system as disclosed herein may include an instrument tray for carrying at least one medical component therein and three-dimensional indicia coupled to at least one side of the instrument tray that includes a recessed panel. Here, the three-dimensional indicia may include a structure symbolic of the at least one medical component within the instrument tray and have a thickness relatively smaller than a depth of the recessed panel. Moreover, the three-dimensional indicia may be of a size and shape that provides external tactile feedback of the structure of the three-dimensional indicia through a sterile wrapping sealing an interior of the instrument tray with the at least one medical component therein. This allows one to identify the at least one medical component within the instrument tray without removing the sterile wrapping.

In these embodiments, the three-dimensional indicia may include an embossed number, letter, geometric shape, or character symbolic of the at least one medical component and have a thickness between 0.25 and 0.3825 inches. Moreover, the depth of the recessed panel may be 0.75 inches, and less than the 0.25 and 0.3825 inch thickness of the three-dimensional indicia. In another aspect of these embodiments, the three-dimensional indicia may be non-removably coupled to the instrument tray. Alternatively, the three-dimensional indicia may be a cutout formed within a sidewall of the instrument tray and have a thickness approximately equal to that of the thickness of the instrument tray sidewall.

Furthermore, the three-dimensional indicia may include multiple three-dimensional indicia that are arranged to generally align with a different row of medical components within the interior of the instrument tray. Here, each of the multiple three-dimensional indicia may include a different structure symbolic of a different medical component within each different row. An edge break that includes a chamfer, a fillet, a radius, or a bevel may smooth out the sides of the three-dimensional indicia to prevent tearing any overlying sterile wrap.

In another alternative embodiment, an instrument tray indicator system as disclosed herein may include an instrument tray having a size and shape for carrying multiple medical components therein, wherein multiple three-dimensional indicia selectively attach to the instrument tray in a pattern representative of a layout of the multiple medical components within an interior of the instrument tray. Here, each of the multiple three-dimensional indicia may have a different structure symbolic of a different one of the multiple medical products within the instrument tray. Moreover, each of the three-dimensional indicia may further be of a size and shape to provide external tactile feedback of the structure of the respective three-dimensional indicia through a sterile wrapping sealing the interior of the instrument tray with the multiple medical components therein, which enables identification of the multiple medical components within the instrument tray without removing the sterile wrapping.

In these embodiments, each of the multiple three-dimensional indicia may be associated with at least one of a perforated sidewall, a front panel, a rear panel, a lid, or a bottom surface of the instrument tray, and include an edge break that includes a chamfer, a fillet, a radius, or a bevel. Moreover, the multiple three-dimensional indicia may each further be of a different embossed number, letter, geometric shape, or character and may selectively removably attach to the instrument tray in modular relation relative thereto. The pattern of the multiple three-dimensional indicia may further include a grid matrix such that the multiple three-dimensional indicia are arranged in a set of rows and columns, similar to and aligned with the layout of the multiple medical components arranged in commensurate rows and columns within the interior of the instrument tray. Each of the multiple three-dimensional indicia may have a thickness of about 0.05 to 1.0 inches. Alternatively, the multiple three-dimensional indicia may be cutouts formed within at least one sidewall of the instrument tray.

In another embodiment, the instrument tray indicator system may include a generally flat or planar base surface that includes one or more unique component identifying indicia formed therein that replicate or geometrically or alphanumerically represents one or more surgical instruments stored within the instrument tray, such as orthopedic implants, when the instrument tray indicator panel is attached thereto.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a perspective view of one type of prior art instrument tray for use in, e.g., orthopedic surgery;

FIG. 2 is a front view of a prior art instrument tray similar to that illustrated in FIG. 1, more specifically illustrating a prior art front panel having one type of known indicia integrally formed therewith;

FIG. 3 is a front elevation view of one embodiment of an instrument tray indicator system as disclosed herein;

FIG. 4 is a front elevation view of an alternative embodiment of an instrument tray indicator system disclosed herein;

FIG. 5 is a top plan view of the instrument tray indicator system of FIG. 2 or FIG. 3;

FIG. 6 is a perspective view of a front panel having the instrument tray indicator system of FIG. 3 selectively coupled thereto;

FIG. 7 is a top plan view of one embodiment a femoral component correlating to an enlarged circular geometric shape indicium;

FIG. 8 is a top plan view of a femoral stem correlating to a rectangular geometric shape indicium;

FIG. 9 is a top plan view of a capital letter “P” correlating to a P-shaped indicium; and

FIG. 10 is a top plan view of a lower case letter “b” correlating to a b-shaped indicium.

### DETAILED DESCRIPTION OF THE PREFERRED EMBODIMENTS

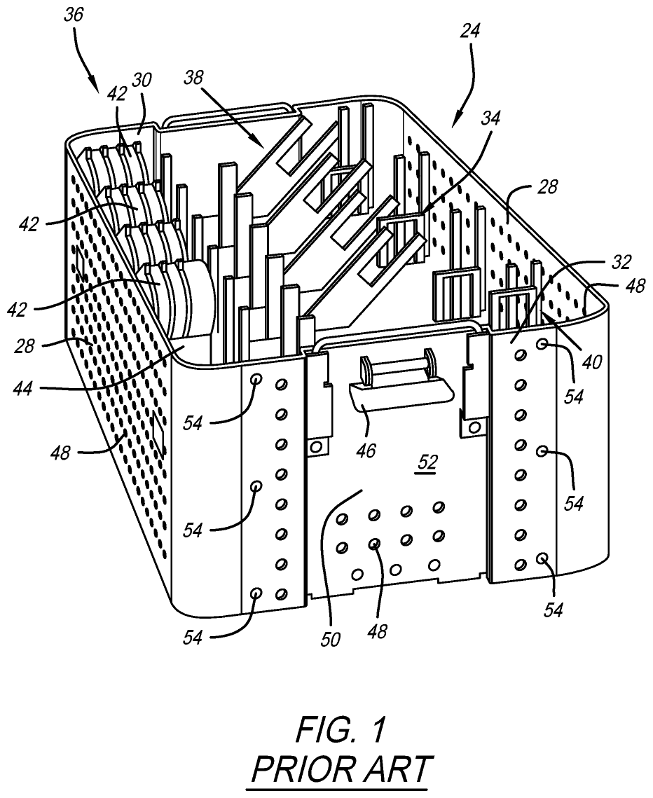

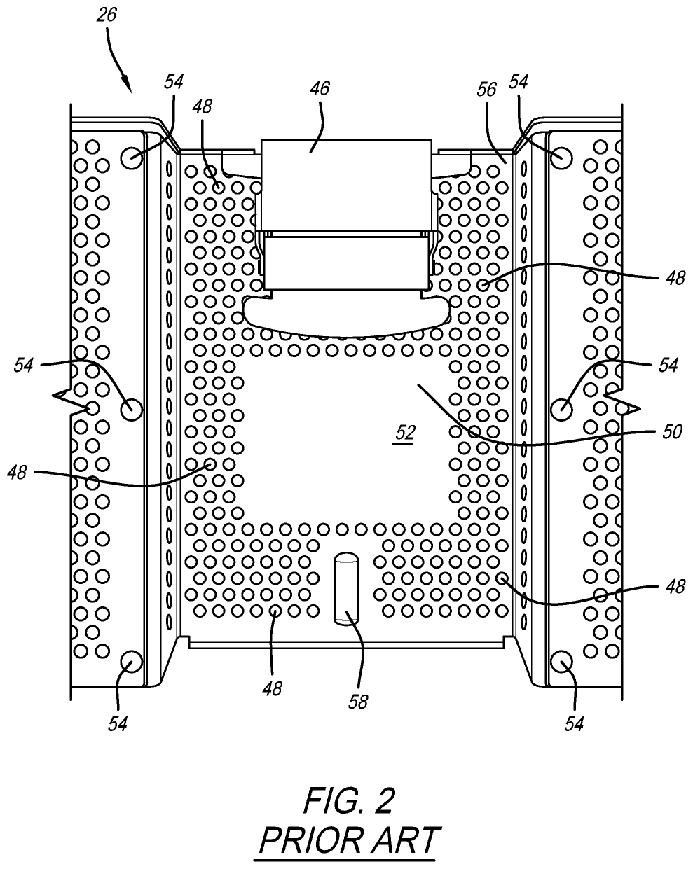

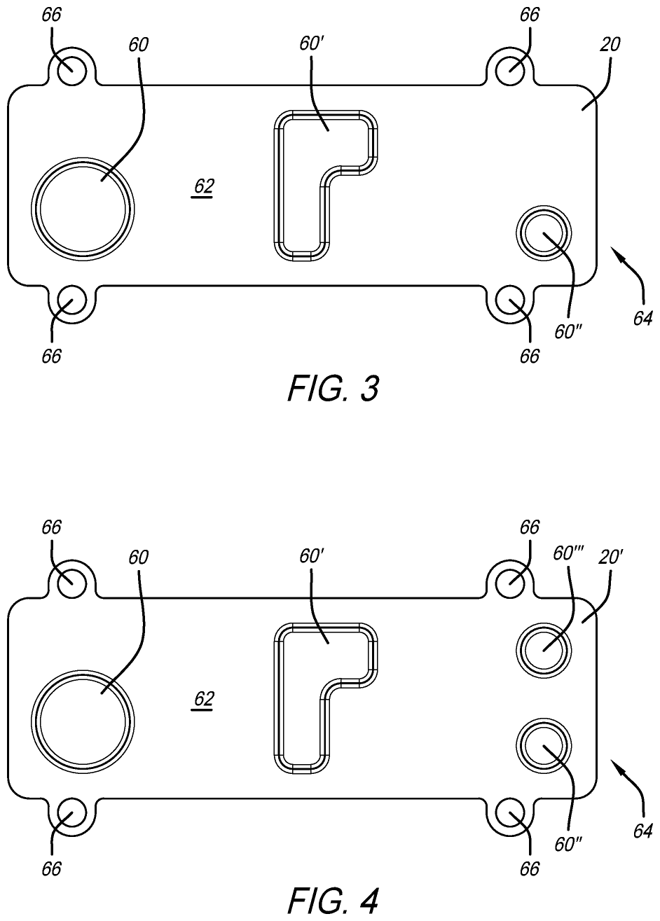

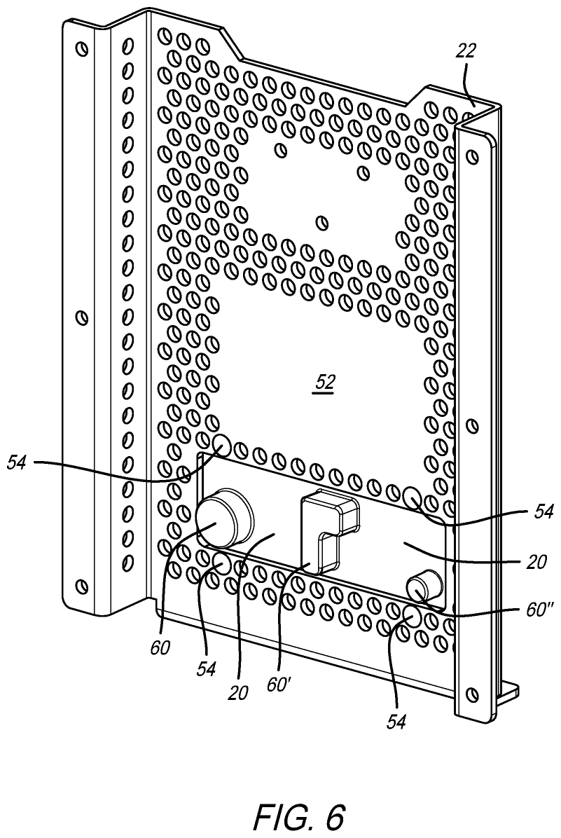

As shown in the exemplary drawings for purposes of illustration, the present disclosure for an instrument tray indicator system is generally illustrated herein with respect to several embodiments, including with respect to the instrument tray indicator panels 20, 20′ in FIGS. 3-6, which may be used in connection with a recessed front panel 22 (FIG. 6) that may be integrated or otherwise used in connection with an instrument tray, such as a prior art instrument tray 24 illustrated in FIG. 1 or an alternative prior art instrument tray 26 illustrated in FIG. 2. While the instrument tray indicator panels 20, 20′ disclosed herein may be compatible with a wide range of instrument trays, the prior art instrument trays 24, 26 illustrated in FIGS. 1 and 2 are exemplary of the type of instrument trays compatible for use with the instrument tray indicator panels 20, 20′, and particularly for use in connection with orthopedic surgical procedures.

More specifically in this respect, FIG. 1 is a perspective view illustrating the prior art instrument tray 24 having a generally rectangular shape formed by a series of perforated upstanding sidewalls 28 formed from a rigid metal material that, together with a prior art recessed rear panel 30 and a prior art recessed front panel 32, form a protective interior 34 for storing one or more medical components therein, such as surgical instruments and/or implants, for use during a surgical procedure (e.g., orthopedic surgery). In the case of the prior art instrument tray 24, the protective interior 34 includes a series of rows, a first row 36, a second row 38, and a third row 40, for storing surgical instruments. For example, the first row 36 is illustrated in FIG. 1 having a series of differently sized inserts 42 secured within the protective interior 34 by way of a set of couplings designed to organize the inserts 42 stored therein by size. Accordingly, since the inserts 42 may be used during knee replacement surgery, the second row 38 and/or the third row 40 may also retain other knee replacement implant components or related surgical instruments that may assist the surgical team in performing orthopedic knee replacement surgery. Of course, similar to the first row 36 storing the series of differently sized inserts 42, the second row 38 and/or the third row 40 may each store common implant components varying in size. As a result, each of the rows 36, 38, 40 may retain a common set of knee implant components unique for each of the rows 36, 38, 40.

The implant components secured within the prior art instrument tray 30 illustrated in FIG. 1 may be enclosed within the protective interior 34 by a lid (not shown). Here, the lid may cooperate with each of the opposing perforated upstanding sidewalls 28 and the prior art recessed rear panel 30 and the prior art recessed front panel 32 to enclose therein the orthopedic components including, e.g., the aforementioned inserts 42. The lid may secure to the prior art instrument tray 42 by way of an externally located handle or clasp 46 coupled to or otherwise integrated with each of the prior art recessed rear panel 30 and/or the prior art recessed front panel 32. As such, when the lid encloses the prior art instrument tray 24, it may be difficult and/or impossible (especially, e.g., when the prior art instrument tray 24 is enclosed with sterile wrapping) to identify the orthopedic components therein, despite the fact that the upstanding sidewalls 28 and/or the prior art recessed rear panel 30 and/or the prior art recessed front panel 32 may include a series of perforations 48 therein, as generally illustrated throughout in each of FIGS. 1 and 2. In fact, the only indicia illustrated with respect to the prior art recessed front panel 32 is a set of logo indicia 50 imparted to a generally planar non-perforated surface 52 thereof that is otherwise unreadable when the prior art instrument tray 24 is enclosed with sterile wrapping.

As is known in the art, the prior art recessed front panel 32 may couple to each of the opposing upstanding perforated sidewalls 28 by a set of rivets 54 illustrated in FIG. 1, and as more specifically illustrated in the enlarged front view of FIG. 2 with respect to the alternative prior art instrument tray 26 and an alternative recessed front panel 56. Similar to the prior art recessed front panel 32 illustrated with respect to FIG. 1, the alternative recessed front panel 56 also includes the perforations 48 formed therein, the clasp 46 for securing the lid over the protective interior 34, and the logo indicia 50 formed within the planar non-perforated surface 52. Although, the alternative prior art recessed front panel 56 illustrated with respect to FIG. 2 also includes a protruding front indicia 58 in the form of a single linear line 58. Such protruding front indicium in the form of the single linear line 58 or the like has been used with respect to prior art devices to indicate the tray number, in this case a first tray. But, even to the extent that such protruding front indicia 58 can be identified through sterile wrapping or with latex gloves prior to or during surgery, such protruding front indicium 58 can be used for nothing more than identifying the number of the tray; and certainly not for identifying any of the surgical components within the tray. Moreover, the protruding front indicia 58 is also formed integrally with the alternative recessed front panel 56, as illustrated in FIG. 2. Thus, to the extent the couplings 44 within the protective interior 34 change, the protruding front indicia 58 does not and cannot otherwise change by way of the fact that it is formed integral with the metal material forming the alternative recessed front panel 56.

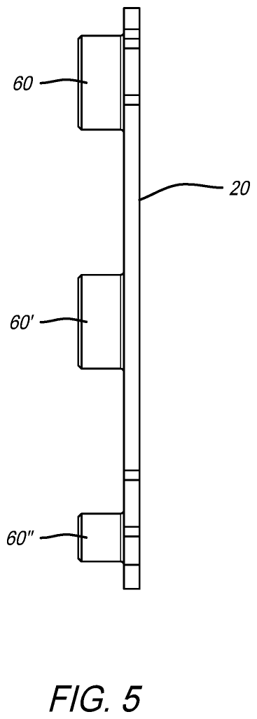

As such, FIGS. 3-5 illustrate a pair of exemplary embodiments of the instrument tray indicator panel 20, 20′ having a set of unique component identifying indicia 60 thereon. Specifically with respect to FIG. 3, one embodiment of the instrument tray indicator panel 20 is illustrated having three of the unique component identifying indicia 60, 60′, 60″ formed thereon, wherein the unique component identifying indicia 60 is in the form of an enlarged circle located in a bottom left hard corner of the instrument tray indicator panel 20, the unique component identifying indicia 60′ is in the form of an uppercase “P” positioned central within the instrument tray indicator panel 20, and the unique component identifying indicia 60″ is in the form of a single relatively smaller circle located in a bottom right hand corner of the instrument tray indicator panel 20. Here, and as best illustrated in the top plan view of FIG. 5, each of the unique component identifying indicia 60, 60′, 60″ generally protrude outwardly from a base surface 62 of the instrument tray indicator panel 20. For example, one or more of the identifying indicia 60, 60′, 60″ may protrude outwardly from the base surface 62 between 0.05 inches and 1.0 inches, and specifically by about 0.25 inches to 0.3825 inches. Alternatively, one or more of the identifying indicia 60, 60′, 60″ may protrude outwardly from the base surface 62 between 0.05 inches and 1.0 inches, and specifically by about 0.25 inches to 0.3825 inches, relative to the panel to which the identifying indicia 60, 60′, 60″ are formed or attach (i.e., in this latter embodiment, the thickness of the base surface 62 may be taken into account when considering the aggregate distance the identifying indicia 60, 60′, 60″ protrude). In another example, the depth of the recessed front panel 32 or the alternative recessed front panel 56 may be about 0.75 inches and the aggregate thickness of the identifying indicia 60, 60′, 60″ may not exceed 0.75 inches such that the identifying indicia 60, 60′, 60″ may provide tactile feedback through sterile wrap covering the tray when tented over the recessed front panel 32 or over the alternative recessed front panel 56 without damaging (e.g., penetrating) the sterile wrap. In this embodiment, the identifying indicia 60, 60′, 60″ effectively remain within the enclave of the recessed front panel 32 or the alternative recessed front panel 56 so as to be positioned in non-contact relation relative to the sterile wrap tented over the recessed front panel 32 or the alternative recessed front panel 56.

Accordingly, each of the unique component identifying indicia 60, 60′, 60″ may be used to uniquely identify the orthopedic implant components stored within each of the respective rows 36, 38, 40 by way of being aligned therewith immediately behind each of the unique component identifying indicia 60, 60′, 60″ when the instrument tray indicator panel 20 is attached to the recessed front panel 22 as illustrated, e.g., in FIG. 6. In other words, the identifying indicia 60 may be designed to provide an indication of the surgical instruments within the row 36, the identifying indicia 60′ may be designed to provide an indication of the surgical instruments within the row 38, and the identifying indicia 60″ may be designed to provide an indication of the surgical instruments within the row 40.

Of course, as discussed in more detail below, the instrument tray indicator panel 20 may include different unique component identifying indicia 60, alternatively, in addition, or in place of those identified above with respect to the relatively large circle formed within a lower left-hand portion of the base surface 62 identified with respect to reference numeral 60, the uppercase “P” formed within the middle of the base surface 62 and identified with respect to reference numeral 60′, and/or the single relatively smaller circle located in a bottom right hand corner of the instrument tray indicator panel 20 and identified with respect to reference numeral 60″.

For example, as illustrated in FIG. 4, the alternative instrument tray indicator panel 20′ may include a pair of the unique component identifying indicial 60″, 60′″ in the form of a pair of the relatively smaller circles formed from a right side 64 of the instrument tray indicator panel 20′. Here, the pair of unique component identifying indicia 60″, 60′″ illustrated in FIG. 4 may be used to identify different orthopedic implant components within the third row 40 stored within the protective interior 34 thereof. In another alternative embodiment, the stacked relationship of the unique component identifying indicia 60″, 60′″ may be used to identify certain implant components within the front half of the third row 40 of the instrument tray (e.g., by way of the unique component identifying indicia 60″) and a different set of components within the back half of the third row 40 of the instrument tray (e.g., by way of the unique identifying indicia 60′″). In other words, this third row 40 may be partitioned into two columns, where the indicia 60″ identifies the first set of components within the first column of the third row 40 and the indicia 60′″ identifies the second (and different) set of components within the second column of the third row 40. This may be especially so when the shape of the unique component identifying indicia 60″ differs from that of the unique component identifying indicia 60′″. As such, the identifying indicia 60 may be positioned to provide tactile feedback in a manner where specific components can be identified within specific sectors of a grid matrix identified by row and column number. The grid matrix may thus earmark specific locations for instruments within the tray, and the indicia 60 may be arranged in a comparable matrix providing external tactile feedback so one can identify specific components within each sector of the grid matrix within the tray through externally applied sterile wrap.

In addition to, or in an alternative of, the unique identifying indicia may be designed to convey information broader than the specific type of components within the tray. Examples here might include conveying information regarding the type of material the components are made from (e.g., cobalt chrome, titanium, etc.), the type of system (e.g., a total knee replacement system or revision knee system), type of joint (e.g., knee, hip, shoulder, etc.), order the tray should be opened, etc. Moreover, the relative location of the indicia may also provide guidance as to whether the indicia are designed to provide broader information or component specific information. In one embodiment, e.g., indicia placed within a top portion of a tray or panel may be designed to convey general information (e.g., component material or system type), while indicia placed along a lower portion of the tray or panel may be designed to convey more specific information (e.g., components being stored within the protective interior 34 of the tray).

Additionally, as illustrated best in FIGS. 3 and 4, the instrument tray indicator panel 20, 20′ may include a set of four mounting apertures 66 in the form of outwardly extending eyelets. Although, in an alternative embodiment, the mounting apertures may be formed as part of the base surface 62 of the instrument tray indicator panel 20 such that the mounting apertures 66 do not outwardly extend therefrom. In either embodiment, the mounting apertures 66 may be of a size and shape commensurate with the perforations 48 formed within the front panel 22 to permit attachment thereto, such as by way of the aforementioned rivets 54 best illustrated in FIG. 6. The rivets 54 may secure the instrument tray indicator panel 20 thereto substantially centered within the recessed portion of the front panel 22 and underneath the planar non-perforated surface 52 where the logo indicia 50 (FIGS. 1 and 2) may be located. In one embodiment, the rivets 54 may permanently attach the instrument tray indicator panel 20 to the front panel 22. Alternatively, the rivets 54, or other connectors as may be known in the art, may be configured to selectively engage the instrument tray indicator panel 20 to the front panel 22. In this latter embodiment, the instrument tray indicator panel 20 may be removed and/or attached to different instrument trays, depending on the contents therein. This latter option may provide more flexibility in identifying the surgical instruments stored by any given tray based on the needs of a particular surgery. In other words, the instrument tray need not necessarily be a one size fits all for purposes of identifying the contents therein by way of the instrument tray indicator panel 20.

The unique component identifying indicia 60 of the instrument tray indicator panels 20, 20′ may be machined from a variety of materials, such as aluminum. Once manufactured, such an aluminum instrument tray indicator panel 20, 20′ may attach to the front panel 22 in the manner illustrated in FIG. 6. In this respect, the embossed symbols forming the unique component identifying indicia 60 may be formed at a height that allows the shape of the identifying indicia 60 to be uniquely identified through sterile wrap and while the medical technician is also wearing latex gloves.

In one embodiment, the unique component identifying indicia 60 may be derived from basic geometric shapes such as the circles as illustrated in FIGS. 3-7, squares, rectangles, triangles, etc. Alternatively, or in addition to, the unique component identifying indicia 60 may be formed to represent alphanumeric characters, such as capital or lowercase letters of the alphabet, numbers, shapes, etc. In this respect, each unique component identifying indicia 60 should be able to convey basic information regarding the contents of the instrument tray through tactile sensation and location, especially for users wearing latex gloves. It may be particularly useful to use the unique component identifying indicia 60 formed into the instrument tray indicator panel 20 to convey location and surgical instrument component information therein when the instrument tray is sealed in sterile wrapping.

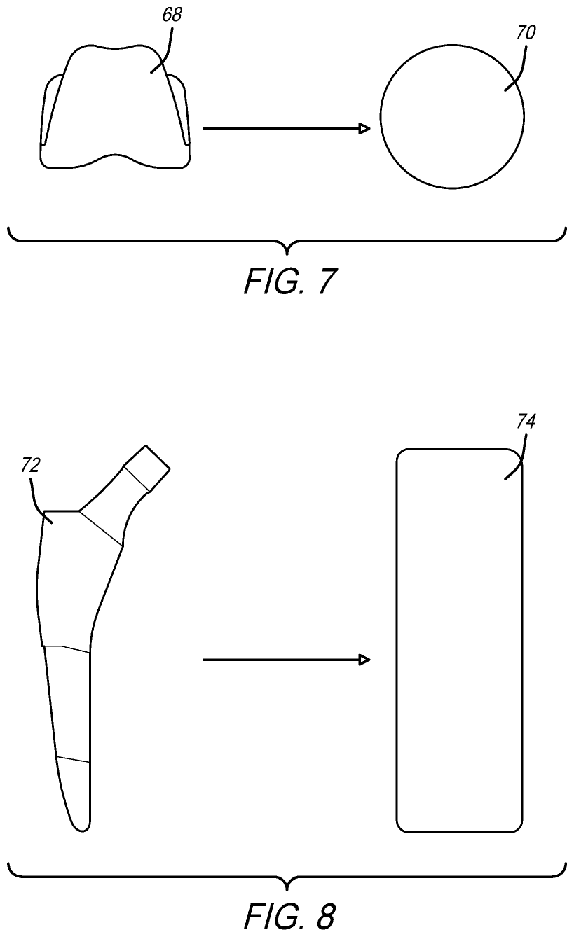

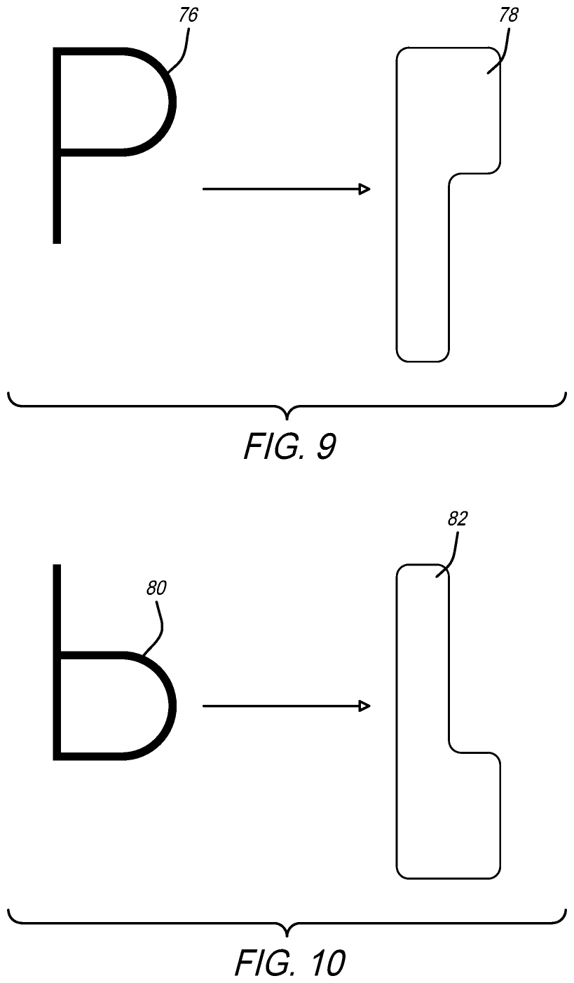

In one example, the unique component identifying indicia 60 may correlate basic geometric shapes or alphanumeric characters with certain implantable joint components the indicia 60 are designed to represent. For example, FIG. 7 illustrates correlating an implantable femoral component 68 with that of a geometric shape that includes an enlarged circle indicium 70. Alternatively, FIG. 8 illustrates correlation of a femoral stem 72 with that of a geometric shape that includes a rectangular geometric shape indicium 74. In additional alternative embodiments, FIG. 9 illustrates correlating a capital letter “P” 76 with that of a P-shaped indicium 78; and FIG. 10 illustrates correlating a lower case “b” 80 with that of a b-shaped indicium 82. In each instance, the enlarged circle indicium 70, the rectangular geometric shape indicium 74, the P-shaped indicium 78, and/or the b-shaped indicium 82 are either geometric shapes or alphanumeric characters that protrude out from the base surface 62 of the instrument tray indicator panel 20, 20′ to provide tactile feedback or sensation in addition to a visual representation of the type of joint component stored within an instrument tray, even when the instrument tray may be enclosed by a lid or otherwise wrapped such that the instruments therein are not directly visible. Furthermore, any of the identifying indicia 60, 60′, 60″, 60′″ and/or indicium 70, 74, 78, 82 may couple directly to the tray (e.g., without the indicator panel 20, 20′) and may further include an edge break that helps prevent the indicia 60, 60′, 60″, 60′″ and/or indicium 70, 74, 78, 82 from tearing, poking through, or otherwise damaging the sterile wrap. The edge break may be a chamfered edge, a filleted edge, a radiused edge, or a beveled edge.

In alternative embodiments, the instrument tray indicator panel 20 and/or the indicia 60 may couple to the instrument tray at different locations (e.g., not necessarily below the planar non-perforated surface 52), such as anywhere along either of the upstanding perforated sidewalls 28, along the perforated rear panel 30, on the lid, or even on the bottom of the instrument tray panel. Attaching additional or alternative instrument tray indicator panels 20 and/or indicia 60 in this manner may increase the level of information conveyed to a medical technician regarding the contents of the instrument tray; and may allow for more specific and unique identification of surgical instruments stored within any surgical tray.

Additionally, while the unique component identifying indicia 60 disclosed herein generally protrude out from the base surface 62 of the instrument tray indicator panel 20, 20′, in alternative embodiments, the unique component identifying indicia 60 may be formed into the base surface 62, thereby forming a geometric or alphanumeric-shaped indentation, aperture, or cutout therein. The same is true with respect to forming any of the identifying indicia 60 and/or the indicium 70 as a shaped indentation, aperture or cutout in any of the perforated sidewalls 28, the recessed rear panel 30, and/or the recessed front panel 32 such that the three-dimensional structure of the indicial 60 and/or the indicium 70 may provide tactile feedback through sterile wrapping by way of tracing the indentation, aperture, or cutout within the panel 20, 20′, and/or the respective perforated sidewall 28, the recessed rear panel 30, or the recessed front panel 32. Here, the identifying indicia 60 and/or the indicium 70 that form a geometric or alphanumeric-shaped indentation, aperture, or cutout in any of the panels 20, 20, the perforated sidewall 28, the recessed rear panel 30, or the recessed front panel 32 may have a thickness of about 0.05 inches to 1.0 inches, and more specifically from about 0.25 inches to 0.3825 inches. Furthermore, the geometric or alphanumeric-shaped indentation, aperture, or cutout may include the aforementioned edge break, which may be a chamfered edge, a filleted edge, a radiused edge, or a beveled edge, albeit internally along the indentation, aperture, or cutout.

Although several embodiments have been described in detail for purposes of illustration, various modifications may be made without departing from the scope and spirit of the invention. Accordingly, the invention is not to be limited, except as by the appended claims.

What is claimed is:

1. An instrument tray indicator system, comprising:
a three-dimensional indicia associated with at least a portion of an instrument tray carrying at least one medical component, the three-dimensional indicia comprising a structure symbolic of the at least one medical component within the instrument tray; and
wherein the three-dimensional indicia comprises a size and shape providing external tactile feedback of the structure of the three-dimensional indicia through a sterile wrapping sealing an interior of the instrument tray with the at least one medical component therein, for identifying the at least one medical component within the instrument tray without removing the sterile wrapping.

2. The instrument tray indicator system of claim 1, wherein the three-dimensional indicia comprises an embossed number, letter, geometric shape, or character symbolic of the at least one medical component.

3. The instrument tray indicator system of claim 1, wherein the instrument tray comprises a multi-sided enclosure and the three-dimensional indicia is associated with at least one side of the multi-sided enclosure.

4. The instrument tray indicator system of claim 3, wherein the multi-sided enclosure includes at least one recessed panel with the three-dimensional indicia attached thereto, the recessed panel having a depth relatively greater than a thickness of the three-dimensional indicia.

5. The instrument tray indicator system of claim 3, wherein the three-dimensional indicia couples to at least one perforation formed in the multi-sided enclosure.

6. The instrument tray indicator system of claim 1, wherein the three-dimensional indicia selectively removably attaches to the instrument tray.

7. The instrument tray indicator system of claim 1, wherein multiple of the three-dimensional indicia are carried by a baseplate selectively attachable to the instrument tray.

8. The instrument tray indicator system of claim 7, wherein each of the multiple three-dimensional indicia are arranged to generally align with a different row of medical components within the interior of the instrument tray.

9. The instrument tray indicator system of claim 8, wherein each of the multiple three-dimensional indicia comprise a different structure symbolic of a different medical component within each different row.

10. The instrument tray indicator system of claim 7, wherein each of the multiple three-dimensional indicia comprise a different structure symbolic of a different medical component in the instrument tray, the multiple three-dimensional indicia being arranged in a grid matrix representative of a layout of the different medical components within the instrument tray.

11. The instrument tray indicator system of claim 1, wherein the three-dimensional indicia comprises a circle symbolic of a femoral component.

12. The instrument tray indicator system of claim 1, wherein the three-dimensional indicia comprises a thickness between 0.05 and 1.0 inches.

13. The instrument tray indicator system of claim 1, wherein the three-dimensional indicia comprises a cutout formed within a sidewall of the instrument tray.

14. The instrument tray indicator system of claim 1, wherein the three-dimensional indicia includes an edge break comprising a chamfer, a fillet, a radius, or a bevel.

15. The instrument tray indicator system of claim 1, wherein the three-dimensional indicia is associated with at least one of a perforated sidewall, a front panel, a rear panel, a lid, or a bottom surface of the instrument tray.

16. An instrument tray indicator system, comprising:
an instrument tray for carrying at least one medical component therein;
a three-dimensional indicia coupled to at least one side of the instrument tray comprising a recessed panel, the three-dimensional indicia comprising a structure symbolic of the at least one medical component within the instrument tray and having a thickness relatively smaller than a depth of the recessed panel; and
wherein the three-dimensional indicia comprises a size and shape providing external tactile feedback of the structure of the three-dimensional indicia through a sterile wrapping sealing an interior of the instrument tray with the at least one medical component therein, for identifying the at least one medical component within the instrument tray without removing the sterile wrapping.

17. The instrument tray indicator system of claim 16, wherein the three-dimensional indicia comprises an embossed number, letter, geometric shape, or character symbolic of the at least one medical component.

18. The instrument tray indicator system of claim 16, wherein the three-dimensional indicia comprises a thickness between 0.25 and 0.3825 inches.

19. The instrument tray indicator system of claim 16, wherein the depth of the recessed panel comprises 0.75 inches.

20. The instrument tray indicator system of claim 16, wherein the three-dimensional indicia is non-removably coupled to the instrument tray.

21. The instrument tray indicator system of claim 16, wherein the three-dimensional indicia comprises multiple three-dimensional indicia arranged to generally align with a different row of medical components within the interior of the instrument tray.

22. The instrument tray indicator system of claim 21, wherein each of the multiple three-dimensional indicia comprise a different structure symbolic of a different medical component within each different row.

23. The instrument tray indicator system of claim 16, wherein the three-dimensional indicia comprises a cutout formed within a sidewall of the instrument tray.

24. The instrument tray indicator system of claim 16, wherein the three-dimensional indicia includes an edge break comprising a chamfer, a fillet, a radius, or a bevel.

25. An instrument tray indicator system, comprising:
an instrument tray having a size and shape for carrying multiple medical components therein;
multiple three-dimensional indicia selectively attachable to the instrument tray in a pattern representative of a layout of the multiple medical components within an interior of the instrument tray, each of the multiple three-dimensional indicia having a different structure symbolic of a different one of the multiple medical products within the instrument tray;
wherein each of the three-dimensional indicia comprise a size and shape providing external tactile feedback of the structure of the respective three-dimensional indicia through a sterile wrapping sealing the interior of the instrument tray with the multiple medical components therein, for identifying the multiple medical components within the instrument tray without removing the sterile wrapping; and
wherein each of the three-dimensional indicia include an edge break comprising a chamfer, a fillet, a radius, or a bevel.

26. The instrument tray indicator system of claim 25, wherein each of the multiple three-dimensional indicia are associated with at least one of a perforated sidewall, a front panel, a rear panel, a lid, or a bottom surface of the instrument tray.

27. The instrument tray indicator system of claim 25, wherein the multiple three-dimensional indicia each comprise a different embossed number, letter, geometric shape, or character and selectively removably attach to the instrument tray in modular relation relative thereto.

28. The instrument tray indicator system of claim 25, wherein the pattern of the multiple three-dimensional indicia comprises a grid matrix of the multiple three-dimensional indicia in a set of rows and columns, which are arranged to generally align with the layout of the multiple medical components arranged in commensurate rows and columns within the interior of the instrument tray.

29. The instrument tray indicator system of claim 25, wherein each of the multiple three-dimensional indicia comprise a thickness of about 0.05 to 1.0 inches.

30. The instrument tray indicator system of claim 25, wherein the multiple three-dimensional indicia comprise cutouts formed within at least one sidewall of the instrument tray.
